(19) United States
(12) Patent Application Publication
(10) Pub. No.: US 2019/0069873 A1
(43) Pub. Date: Mar. 7, 2019

(54) AUSCULTATION OF A BODY

(71) Applicants: Ryan J. Copt, Port St. Lucie, FL (US); Joseph G. Butera, III, Stuart, FL (US); Robert J. Summers, III, Port St. Lucie, FL (US)

(72) Inventors: Ryan J. Copt, Port St. Lucie, FL (US); Joseph G. Butera, III, Stuart, FL (US); Robert J. Summers, III, Port St. Lucie, FL (US)

(21) Appl. No.: 16/116,334

(22) Filed: Aug. 29, 2018

Related U.S. Application Data
(60) Provisional application No. 62/554,668, filed on Sep. 6, 2017.

(51) Int. Cl. A61B 7/04; H04R 1/46; H04R 1/28; A61B 7/02

(52) U.S. Cl. CPC A61B 7/04; A61B 7/023; H04R 1/2876; H04R 1/46

(57) ABSTRACT

Acoustic listening methods, devices, and systems herein relate to auscultation of a body. An auscultation device as disclosed herein can be operable to function within a cavity of the body, and can operate in conjunction with other auscultation devices, including with eternal auscultation devices. Individual devices and grouped devices can operate with the addition of a computing device.

### CLAIM OF PRIORITY

The present Non-Provisional patent application claims priority pursuant to 35 U.S.C. Section 119(e) to a currently pending and prior filed Provisional patent application, namely, that having Ser. No. 62/554,668 filed on Sep. 6, 2017, the contents of which its incorporated herein by reference in its entirety.

### BACKGROUND OF THE INVENTION

### Field of the Invention

The present invention relates to devices, systems, and methods for auscultation of a body.

### Description of the Related Art

Auscultation, or the term for listening to the internal sounds of a body, is of great importance to many disciplines, such as the medical fields. For example, auscultation of a body, such as the body of a patient, assists a medical professional in the diagnosis of ailments that may affect the patient. Such may be traditionally achieved with a stethoscope, which may use a wide bell and/or a diaphragm to listen to a narrow range of low frequency acoustic signals, such as those associated with patient's heartbeat. However, such approaches are fundamentally inadequate for many other diagnostic purposes, such as receiving acoustic signals associated with higher frequency signals. Also, medical professionals are increasingly in need of more sensitive and more precise means of diagnosing patients. Current approaches, while effective, are subject to inherent limits based on past technologies.

Accordingly, what is needed in the art is a device structured to more sensitively receive acoustic signals in a wide band of frequencies, including but not limited to high-frequency sounds. Such acoustic signals may not be easily detectable by traditional devices located at the skin's surface. Additionally, a medical professional may not be as capable of detecting and analyzing all the auscultation data detected by a device. A means of automatically detecting, storing, and analyzing auscultation, either alone or in conjunction with a medical professional, is needed.

Further, what is needed in the art is a system incorporating such a device. Such a system may incorporate the device to facilitate in the diagnosis of patients and/or other medical procedures carried out by medical professionals. Such a system would utilize the acoustic signals received by the device, and process the signals to assist in detection of, for example, disorders of the gut, the joints, the lungs, blood flow, or swallowing.

### SUMMARY OF THE INVENTION

Some or all of the above needs and/or problems may be addressed by certain embodiments of the disclosure. Certain embodiments may include systems, devices, and methods for auscultation of a body. According to one embodiment of the disclosure, there is disclosed a system. The system can include an internal auscultation device for use within a cavity of the body. The system can also include an external auscultation device for use outside the body. And the system can also include an external computing device in communication with the internal auscultation device and the external auscultation device.

According to another embodiment of the disclosure, there is disclosed a method. The method can include configuring an internal auscultation device to be disposed within a cavity of a body. The method can include configuring the device with one end structured to receive an acoustic signal. The method can also include configuring the device to include a transducer capable of converting an acoustic signal into an electrical signal.

According to another embodiment of the disclosure, there is disclosed a device. The device can include an exterior molding configured to fit within a cavity of a body, and the exterior molding can include an open end and a closed end. The exterior molding can include a chamber structured to receive an acoustic signal. The device can also include a transducer capable of converting an acoustic signal into an electrical signal.

These and other objects, features and advantages of the present invention will become clearer when the drawings as well as the detailed description are taken into consideration.

### BRIEF DESCRIPTION OF THE DRAWINGS

For a fuller understanding of the nature of the present invention, reference should be had to the following detailed description taken in connection with the accompanying drawings in which:

FIG. 1 illustrates an example system of auscultation of a body according to one embodiment of the disclosure.

FIG. 2 is a flow diagram of an example method for auscultation of a body.

FIG. 3A illustrates a cross-section view of an example device for auscultation of a body, according to one embodiment of the disclosure.

FIG. 3B illustrates a view into an open end of an example device for auscultation of a body, according to one embodiment of the disclosure.

Like reference numerals refer to like parts throughout the several views of the drawings.

### DETAILED DESCRIPTION OF THE PREFERRED EMBODIMENT

Illustrative embodiments of the disclosure will now be described more fully hereinafter with reference to the accompanying drawings in which some, but not all, embodiments of the disclosure are shown. The disclosure can be embodied in many different forms and should not be construed as limited to the embodiments set forth herein, rather, these embodiments are provided so this disclosure will satisfy applicable legal requirements.

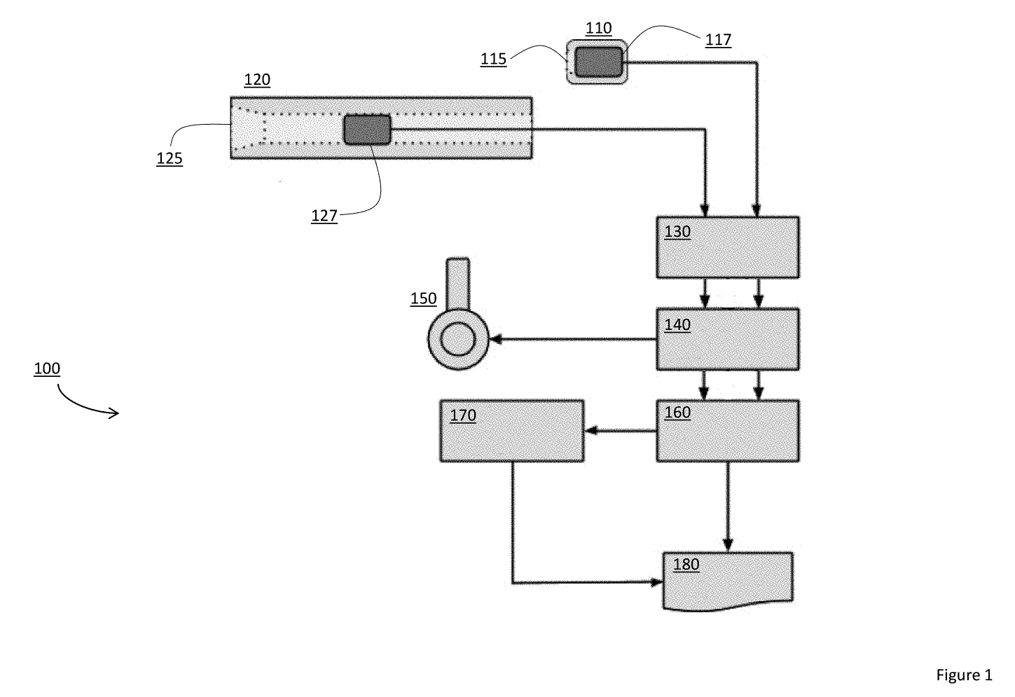

Certain embodiments disclosed herein relate to systems for auscultation of a body. Referring to FIG. 1, depicted is an example system 100 for auscultation of a body. System 100 can be used in conjunction with method 200 and device 300. System 100 can capture acoustic signals and process the acoustic signals for analysis and diagnosis by a human or by a pattern recognition engine 170, or both. The system 100 can detect higher frequency signals that are not normally used in clinical diagnosis. One goal of system 100 is to provide bedside screening for dysphagia or other disorders related to the head and neck region. The system 100 can use a microphone enclosure for capturing sounds 110 from cavities, such as ear and nasal cavities; and the system 100 can also use a tuned resonant structure enclosing a microphone 120. The signal can be fed to an adaptive audio signal processing system capable of normalizing audio level and frequency content for the desired sounds. The audio processing can include dynamic range control and frequency filtering. System 100 can be used for biological screening and analysis, among other applications. System 100 can be used by a bedside clinician or caretaker to administer a screening test or to do more in-depth analysis. System 100 can include an acoustic capture device 110 for use within a cavity of a body. In some embodiments, the cavity can be an ear or nasal cavity. System 100 can include an internal auscultation device 110. Internal auscultation device 110 can include an exterior molding, and the exterior molding can include a proximal and a distal end. The proximal end can include an opening 115 that is dimensioned and configured for acoustic engagement within a cavity of the body. Acoustic engagement within a particular cavity can depend on the cavity, for example, one operative orientation for device 110 is when opening 115 is pointed toward the inside of the cavity while the distal end of device 110 is pointed toward outside the cavity. Internal auscultation device 110 can also include one or more chambers configured within the exterior molding, and the chambers can be collectively structured to receive an acoustic signal, for example, when opening 115 is pointed toward a source of the acoustic signal. Device 110 can include one or more transducers 117 disposed within the chambers and configured to receive the acoustic signals. Transducer 117 can then convert the acoustic signals to electrical signals, and the electrical signals can be used by other components of system 100. System 100 can include another auscultation device 120. Device 120 can be configured and disposed for use outside a body, and can be structured to receive an acoustic signal. The acoustic signal received by external auscultation device 120 can be different from the signal received by device 110, or the signals can be the same, or some components of the signals can be the same while other components of the signals can be different. Device 120 can include a proximal end and a distal end, and the proximal end can include opening 125 for orientation toward a source of an acoustic signal. Device 120 can include an exterior molding configured to dampen ambient noise. Device 120 can also include multiple chambers collectively structured to receive the acoustic signal. Device 120 can also include at least one transducer 127 operatively situated within the exterior molding of device 120 and configured to convert acoustic signals to electrical signals. The electrical signals can then be used by other components of system 100. The system 100 can also include an external computing device for processing, analysis, and/or display of the information associated with acoustic signals. In some embodiments, the external computing device can include a microphone preamplifier 130. Microphone preamplifier 130 can receive electrical signals from devices 110 and 120 and can boost those signals for more efficient processing of the signal information by the other components of system 100. Microphone preamplifier 130 can be communicatively coupled to a digital signal processor (DSP) 140. DSP 140 can be operable to process electrical signals received from microphone preamplifier 130 as well as from all auscultation devices included in system 100, for example, one or more internal devices 110 and one or more external devices 120. In some embodiments, signals may be received by the DSP 140 directly from devices 110 and/or 120 without the signals first being boosted by microphone preamplifier 130.

In some embodiments, device 110 or device 120 or both can include an external layer that dampens ambient noise levels. This noise-dampening layer can be useful in reducing the unwanted acoustic information received by transducers 117 and/or 127. In this way, the acoustic signals ultimately processed by transducers 117 and/or 127 can then be more accurately directed from the desired source of acoustic signals. In some embodiments, system 100 can include an audio output to one or more headphone devices 150. System 100 can include a time and frequency analysis 160 which can be displayed on a screen 180 or analyzed for pattern recognition 170, or both.

In some embodiments, device 110 and/or device 120 can be configured with a bell structure, for example, with a wider opening at the proximal end. The bell structure can be vented into the primary opening 115 and/or 125 via a small hole within one of the internal chambers. The diameter of the hole is constructed to allow a desired amount of low frequency content into a high frequency primary opening. When the device 110 or 120 is placed against a body, high frequencies can be captured in the primary opening 115 or 125. Lower frequency content can be simultaneously, and separately, captured in the larger concentric bell. Lower frequencies can be captured more efficiently so they are attenuated before being “mixed” with high frequency content. The attenuation and mixing can be accompanied by allowing low frequency content to pass into the high frequency opening through a small diameter hole. The composite sound can be captured by a single microphone in a microphone chamber, and this chamber can be sealed with a cap.

In some embodiments, devices 110 and/or 120 can include exterior moldings with layering of multiple, dissimilar materials. This layering can, among other things, create an impedance barrier to vibrational energy and dampen resonant characteristics of denser materials. In a basic form, layering could require three materials layered with each other, and the layering can be expanded to include more layers which can increase performance if, for example, the material for each layer is of a different density than its neighboring material(s). In one embodiment, the layering can include a first material that constitutes the outer body of device 110 and/or 120. This outer body can be a rigid material of moderate to high density such as, but not limited to, aluminum, steel, stainless steel, or any number of high density plastics. The outer body of device 110 and/or 120 can then be shrouded in a layer of a second material on all faces except for the proximal end oriented toward the source of an acoustic signal. The second material can be pliable such as composed of putty, gel rubber, or foam. This second material can impede the transmission of vibrational energy and serve to dampen resonant characteristics of the first material. This second layer can, in turn, be shrouded by a third material on all faces except the proximal end. The third material can be similar to the first material in a property of rigidness and of moderate to high density. In some embodiments, the first and third materials are different from each other even though they may share some qualities and properties. Configuring the materials to be dissimilar can increase performance of device 110 and/or 120. However, device 110 and/or 120 still performs as desired if the first and third materials are the same but, in some scenarios, that performance may be decreased. The usage of multiple, dissimilar layers works to create multiple impedance barriers which can significantly reduce the amount of vibrational energy transmitted through device 110 and/or 120. The layers also can serve to dampen resonant characteristics of the rigid materials.

In some embodiments, for example for purposes of sanitization, acoustic, and/or functional requirements of some stethoscope applications, a diaphragm can be attached, such as temporarily, to the outermost layer of device 110 and/or 120 at the proximal end. The diaphragm can be molded from a single piece of plastic or it can be constructed using multiple materials, depending on the desired acoustic or other requirements. Some benefits of a disposable diaphragm include economical production as a single piece of plastic, construction with varying thicknesses or materials to provide alternative acoustic characteristics, sanitary barrier to microphone and interior elements of a stethoscope, provided through hands-free attachment packaging (similar to otoscope ends) so a new diaphragm can be attached without a user touching the diaphragm before use, facilitating auscultation over clothing for cases where that scenario is required, and providing mechanical isolation of the microphone housing from the body. The outer ring of the diaphragm can be configured to fit over the outer shell of device 110 and/or 120 at the proximal end. This ring can be rigid and can include a locking mechanism to prevent the diaphragm from falling off during use. A seal can be created with same or different materials to provide an acoustic (air-tight) closure over the proximal end. The seal can also provide a mechanical stand-off so the diaphragm does not come in contact with the primary inner bell structure as the seal can be made with the outer shell only. The diaphragm can be within 0.1 millimeters and 0.75 millimeters thick to provide good isolation while allowing vibrations from the body to pass through with minimal impedance. The entire diaphragm assembly need not touch any part of the inner microphone housing thereby providing, among other things, mechanical isolation from environmental and other unwanted noise sources.

In one embodiment, system 100 can be used for obtaining acoustic information relating to the physiology of a person's swallowing. System 100 can be used to monitor the person's swallowing over a period of time or during fluoroscopy. In one embodiment of the swallowing monitoring, an external auscultation device 120 can be attached to a person via straps and/or an adhesive. This external auscultation device 120 can be placed at the midline of the neck, for example, inferior to the thyroid cartilage and superior to the jugular notch. In this embodiment, an internal auscultation device 110 can be placed in an ear canal of the person and held in place by the foam or elastic material comprising the device's 110 exterior molding. This placement could also be within a nasal cavity. In this and other embodiments, auscultation devices 110 and 120 can be amplified by microphone preamplifier 130 and processed by DSP 140 and analyzer 160 and recognition device 170 before the data is displayed to a clinician via display 180. The information received by separate auscultation devices, for example by devices 110 and 120, can be carried by separate cables and/or the separate signals can be carried by a single multi-channel cable. In one embodiment, some or all of the components such as the microphone preamplifier, DSP, time/frequency analysis, pattern recognition system, and display can be contained within a single device, such as a handheld device. The handheld device can include one or more light-emitting diodes (LEDs) to denote the presence or absence of some information, or to convey other information to a clinician, for example, and can be included in the display 180. In this or other embodiments, the display 180 can include a screen, such as a touchscreen to both convey information to a user as well as receive input from a user.

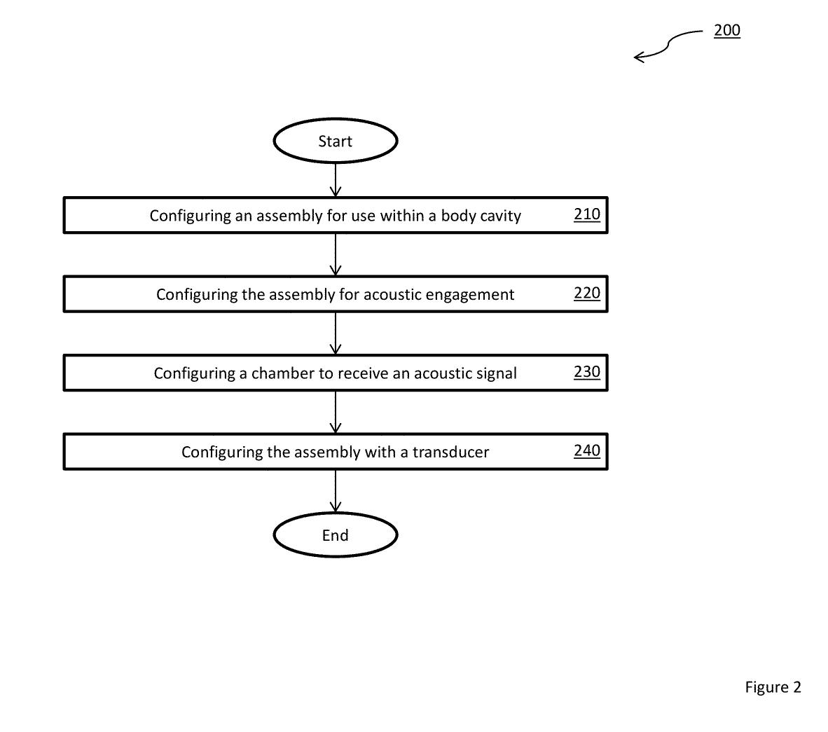

According to another embodiment of the invention, and with reference to FIG. 2, depicted is a flow diagram of an example method 200 for auscultation of a body. The method 200 can be utilized in association with various systems and devices, such as system 100 and device 300. The method 200 can begin at block 210. At block 210, a device can be assembled to be used for auscultation within a cavity of the body, for example, within an ear or nasal cavity. The assembly can be configured to fit within the cavity. In one embodiment, an elastic material can be used in the assembly, and the elastic material can compress to fit within differently sized cavities and still create a seal between the cavity and the ambient air. In one embodiment, the elastic assembly can include a foam or foam-like material. As block 220, the auscultation assembly can be configured for acoustic engagement of the body. For example, the assembly can include one end, in some cases called a proximal end, that includes an airway opening. In some embodiments, the assembly can include multiple openings. The assembly can be configured so an opening of the assembly, for example the proximal end, can point toward the interior of the cavity. The opening can receive an acoustic signal originating from within the body. For example, the acoustic signal can travel from its origin within the body, through the cavity, and then through the opening of the assembly. At block 230, the assembly can be configured with a chamber to receive the acoustic signal. The chamber can be designed to allow the acoustic signal to travel the entire distance of the chamber, through and then away from the proximal end and toward the distal end, while preserving the integrity of the acoustic signal. In one embodiment, the chamber can have parallel “walls” where the chamber is essentially cylindrically shaped with virtually the same distance between the walls at the proximal end as there is at the distal end. In another embodiment, the chamber can be bell-shaped such that the distance between the walls is greater at the base of the opening (proximal end) than at the other end of the chamber (distal end). The chamber can also include subchambers, some of which are bell-shaped and some of which are cylindrical, or the subchambers can be all of a similar shape. At block 240, the assembly can be configured with a transducer. An interior chamber of the assembly can be designed to receive and hold a transducer, and the interior chamber can be configured to enable and promote operation of the transducer. The transducer can reside at the end of the assembly opening such that the transducer is the first object encountered by an acoustic signal after entering the opening at the proximal end. The transducer can convert the acoustic signal into an electrical signal, and the electrical signal can then be used in support, analysis, and/or storage of information for the assembly.

Method 200 may optionally end following block 240.

The operations described and shown in method 200 of FIG. 2 may be carried out or performed in any suitable order as desired in various embodiments of the disclosure, and method 200 may repeat any number of times. Additionally, in certain embodiments, at least a portion of the operations may be carried out in parallel. Furthermore, in certain embodiments, fewer or more operations as illustrated in FIG. 2 may be performed.

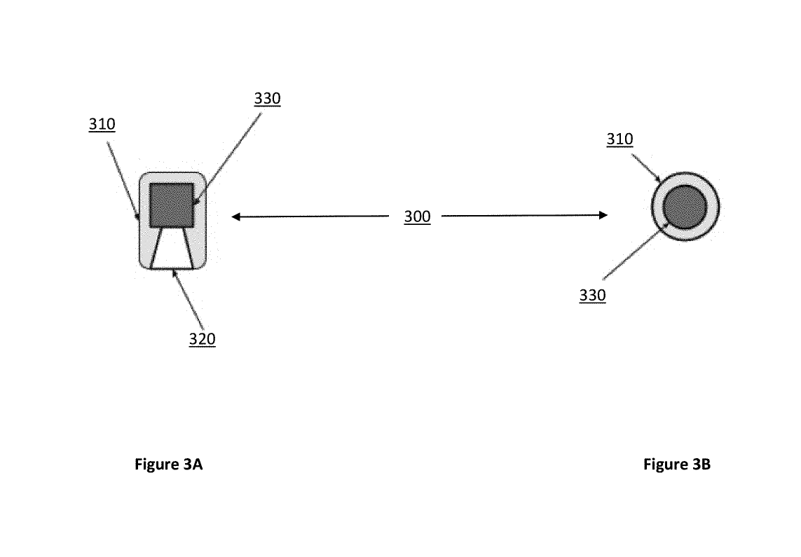

According to another embodiment of the invention, and with reference to FIGS. 3A and 3B, disclosed is a device 300 for auscultation of a body. Device 300 can include an exterior molding 310. The exterior molding 310 can be formed as a single piece, for example, through casting, forming, or 3-D printing. The exterior molding 310 can be of a shape to dampen acoustic signals, and the material composing the molding 310 can be an acoustic dampening material. In this way, device 300 can reduce the reception of unwanted acoustic signals and channel the desired internal acoustic signals from inside the body through opening 320 and to transducer 330. The material used in the molding 310 can include a degree of elasticity so that device 300 can be compressed, if necessary, to fit inside the cavity, for example an ear or nasal cavity, and then naturally expand by retaking its original shape, creating a seal between the walls of the cavity. The shape of the molding 310 can be customized to fit within the particular cavity, while still allocating space within molding 310 for a transducer 330 and acoustic opening 320. In one embodiment, molding 310 can be cylindrically shaped with rounded edges. Molding 310 can include a proximal end with an opening 320 for acoustic engagement within the cavity. The opening 320 can be oriented toward the inside of the cavity and directly receive the acoustic signal originating from inside the body. Opening 320 can be shaped to receive the acoustic signal. Opening 320 can also convey the acoustic signal from the proximal end in through to the distal end of molding 310. The passageway beginning at opening 320 can be cylindrical in shape, among other possible shapes. In one embodiment, the passageway can be bell-shaped having, for example, a wider diameter at opening 320. In other embodiments, the passageway can be divided into chambers to facilitate the passage of the acoustic signal, and/or to accommodate other components of device 300. Device 300 can also include one or more transducers 330. The transducer(s) 330 can convert the acoustic signals into electrical signals.

In some embodiments, device 300 can include an external computing device. The external computing device can receive communication from transducer 330, for example, through wireless network communication. In one embodiment, transducer 330 receives a body's acoustic signal via opening 320. Transducer 330 can then convert that acoustic signal into an electrical signal for, among other reasons, more efficient transmission of the acoustic signal to a remote location. In some embodiments the electrical signal originating at the transducer 330 can be received by a microphone preamplifier. The microphone preamplifier can boost the electrical signal for continued transmission. In some embodiments, device 300 can include a DSP. The DSP can receive the signal from the microphone preamplifier, or from the transducer 330, or both. The DSP can include processing that incorporates audio frequency dynamic range control and/or equalization. The audio processing can also include frequency filtering. Device 300 can perform time and frequency analysis on the audio signal. In some embodiments, a time and frequency analysis can be used to perform a pattern recognition evaluation of the frequency, intensity, and/or time. In some embodiments, device 300 includes a display. The display can output, for example, the pattern recognition evaluation, the time and frequency analysis, and/or other information pertaining to the auscultation. The display can include one or more light-emitting diodes (LEDs) for displaying information. The display can also include a screen for displaying information. In some embodiments, the display can include an interactive touch screen.

In some embodiments, device 300 can include multiple assemblies containing a molding 310 with a transducer 330. Some or all of the assemblies can transmit their respective acoustic information to a DSP. Some of the multiple assemblies can be designed for internal (e.g. within a cavity) placement, and some of the assemblies can be designed for external (e.g. outside a cavity) placement. In some embodiments, device 300 can include one or more headphone outputs to enable listening to the signals that have been captured. The headphone outputs can be connected to the DSP. The headphone outputs can be standard headphones and the headphone outputs can be purpose-built to work with device 300 for auscultation of a body.

One example of headphone outputs can provide hearing protection in high-noise environments while simultaneously providing high quality, electronic sound with situational/directional integrity of the sound. These headphone output embodiments can also be utilized in other applications, such as in extremely loud ambient noise scenarios, in addition to use in the immediate disclosure. The outputs can include a circumaural muff designed to reduce ambient sound by at least 30 decibels. The headphone outputs can include one or more in-ear “buds.” The buds can use foam eartips and a fully sealed system to provide additional ambient noise rejection of 20 decibels and higher. The buds can include a speaker for audio playback. The outputs can also include electronic voice communication input, for example, a wired audio connection or wireless audio receiver, such as Bluetooth, 2.4 GHz, etc. The outputs can also include situational awareness microphone input. One embodiment of the situational awareness microphone input can include at least one microphone mounted on the outside of each circumaural earcup, and each microphone can be positioned to face forward relative to the wearer's face. Each microphone may be contained within a manifold designed to mimic mechanical filtering of a human ear, and the output of each microphone can feed a pre-amplifier. The headphone outputs can also include DSP and amplification. The DSP can receive the electronic sound or voice communication and the preamplified situational awareness. The DSP is programmed to provide increased speech intelligibility for voice communications and create a natural, realistic recreation of the directional and situational (e.g. outside world) on the microphone signal. The output signal from the DSP can be fed into an amplifier which drives the speakers in the in-ear buds. The in-ear buds can be tethered to the interior of the earcups such that no external wires need exit the earcups and compromise the seal of the muffs against the wearer's head. The tether wire (which can carry the signal to speakers in the buds) can be governed by a spring-loaded or ratcheting take-up reel. Inclusion of the take-up reel could allow an unusually long tether wire to be used. A longer tether wire can allow for easier placement of the buds into the user's ears. Any excess length of wire could then be automatically (or at the press of a button) coiled back when the muffs are positioned on the user's head. This could eliminate the need to bunch up the excess wire inside the earcup which makes the system easier to put on. Eliminating excess wire bunch can also provide superior comfort to the user. The DSP can be programmed in multiple ways, and the signal could be affected as described, for example, in U.S. Pat. Nos. 8,160,274 and/or 9,264,004, for the purpose of, among other things, to effectively and measurably increase the intelligibility of the incoming sound signal, including human speech. Some benefits of using this type of method can include: superior frequency response control allowing for natural and realistic representation of real-world acoustic environments; ability to limit extremely loud transient sounds to safe levels without any loss of or artifacting of other environmental sounds (e.g. If a person is speaking and a gunshot occurs nearby, the gunshot can be limited to a safe level while the person's voice would be perceived to remain at a consistent level.); and/or if coupled with the aforementioned microphone manifold, this processing can achieve a perfect recreation of the directionality of environmental sounds on all axes. Output signals from the DSP can be combined in multiple, different ways for different embodiments of the system. For example, the DSP can provide a user with level control by which the mix between voice communication and situational awareness can be continually adjusted. Also, voice communication may always be enabled with the situational awareness muted. The situational awareness can then be turned on by use of a momentary switch, for example, located on an external portion of one or both ear muffs. This could allow a push-to-talk type of feature for communicating with persons within the environment. Additionally, the DSP can, by default, have both voice communication and situational awareness turned on, while being programmed with a threshold for automatic muting and unmuting of the situational awareness microphones. Yet another example is a voice communication input can be combined with or replaced by an additional wired or wireless audio input designed to carry entertainment, e.g. music, etc. If the two are combined into a single channel, the DSP can be programmed for multiple modes in order to, among other things, provide superior speech intelligibility for voice and digital audio enhancement for entertainment. If the two remain in separate channels, they could be processed separately by the DSP for their respective purposes.

Since many modifications, variations and changes in detail can be made to the described preferred embodiment of the invention, it is intended that all matters in the foregoing description and shown in the accompanying drawings be interpreted as illustrative and not in a limiting sense. Thus, the scope of the invention should be determined by the appended claims and their legal equivalents.

What is claimed is:

1. A system for auscultation of a body, said system comprising: an internal auscultation device comprising:
an exterior molding comprising a proximal end and a distal end, said proximal end including an opening dimensioned and configured for acoustic engagement within a cavity of the body when said exterior molding is disposed in a first operative orientation;
said exterior molding including at least one chamber disposed therewithin collectively structured to receive a first acoustic signal at least when said exterior molding is disposed in the first operative orientation;
at least one transducer at least partially disposed in a corresponding one of said at least one chamber and structured to convert the first acoustic signal into a first electrical signal;
an external auscultation device disposed for use outside the body, and structured to receive a second acoustic signal at least when said external auscultation device is disposed in a second operative orientation, said external auscultation device comprising at least one external transducer structured to convert the second acoustic signal into a second electrical signal; and
an external computing device communicably attached to the internal auscultation device and to the external auscultation device, said external computing device comprising a digital signal processor (DSP) for processing at least one of the first electrical signal and the second electrical signal.

2. The system as recited in claim 1, wherein at least one of the internal auscultation device and the external auscultation device comprises a layer comprising an acoustically dampening material.

3. The system as recited in claim 1, wherein said opening is dimensioned and configured for acoustic engagement within an ear cavity.

4. The system as recited in claim 1, wherein said opening is dimensioned and configured for acoustic engagement within a nasal cavity.

5. The system as recited in claim 1, further comprising at least one of a microphone preamplifier, a time and frequency analysis, a pattern recognition processor, and an audio output.

6. The system as recited in claim 1, further comprising a handheld device, said handheld device housing the DSP.

7. The system as recited in claim 6, wherein said handheld device further comprises at least one light-emitting diode (LED).

8. The system as recited in claim 1, wherein said handheld device further comprises a screen display.

9. A method of auscultating a body, said method comprising:
configuring an assembly with an exterior molding for disposition within a cavity of the body;
configuring said exterior molding with a proximal end and a distal end, said proximal end including an opening dimensioned and configured for acoustic engagement within the cavity when said exterior molding is disposed in an operative orientation;
configuring said exterior molding to include at least one chamber disposed therewithin collectively structured to receive an acoustic signal at least when said exterior molding is disposed in the operative orientation; and
configuring said assembly with at least one transducer, the at least one transducer at least partially disposed in a corresponding one of said at least one chamber and structured to convert the acoustic signal into an electric signal.

10. The method as recited in claim 9, further comprising configuring said exterior molding to be comprised of an acoustically dampening material.

11. The method as recited in claim 9, wherein said assembly is configured for disposition within an ear cavity.

12. The method as recited in claim 9, wherein said assembly is configured for disposition within a nasal cavity.

13. The method as recited in claim 9, further comprising digitally processing the acoustic signal, via an external computing device, said external computing device comprising a digital signal processor (DSP) communicably attached to said at least one transducer.

14. The method as recited in claim 13, further comprising at least one of preamplifying the acoustic signal, digitally processing the acoustic signal for pattern recognition, analyzing the acoustic signal according to duration and frequency, and outputting the acoustic signal to an audio device.

15. The method as recited in claim 13, further comprising housing the DSP in a handheld device.

16. The method as recited in claim 15, wherein said handheld device further comprises at least one light-emitting diode (LED).

17. The method as recited in claim 16, wherein said handheld device further comprises a screen display.

18. The method as recited in claim 9, further comprising configuring an external auscultation device disposed for use outside the body.

19. The method as recited in claim 9, further comprising communicably attaching said external auscultation device to said external computing device.

20. A device for auscultation of a body, said device comprising:
an exterior molding comprising a proximal end and a distal end, said proximal end including an opening dimensioned and configured for acoustic engagement within a cavity of the body when said exterior molding is disposed in an operative orientation;
said exterior molding including at least one chamber disposed therewithin collectively structured to receive an acoustic signal at least when said exterior molding is disposed in the operative orientation; and
at least one transducer at least partially disposed in a corresponding one of said at least one chamber and structured to convert the acoustic signal into an electrical signal.

21. The device as recited in claim 20, wherein the exterior molding is comprised of an acoustically dampening material.

22. The device as recited in claim 20, wherein said opening is dimensioned and configured for acoustic engagement within an ear cavity.

23. The device as recited in claim 20, wherein said opening is dimensioned and configured for acoustic engagement within a nasal cavity.

24. The device as recited in claim 20, further comprising an external computing device, said external computing device communicably attached to the at least one transducer, said external computing device comprising a digital signal processor (DSP).

25. The device as recited in claim 24, further comprising at least one of a microphone preamplifier, a time and frequency analysis, a pattern recognition processor, and an audio output.

26. The device as recited in claim 24, further comprising a handheld device, said handheld device housing the DSP.

27. The device as recited in claim 26, wherein said handheld device further comprises at least one light-emitting diode (LED).

28. The device as recited in claim 26, wherein said handheld device further comprises a screen display.

29. The device as recited in claim 20, further comprising an external auscultation device, said external auscultation device disposed for use outside the body.

30. The device as recited in claim 29, wherein said external auscultation device is communicably attached to said external computing device.
